(19) United States
(12) Patent Application Publication
(10) Pub. No.: US 2025/0342913 A1
(43) Pub. Date: Nov. 6, 2025

(54) METHOD FOR MONITORING A PRODUCTION CONSIDERING AN ENVIRONMENTAL IMPACT

(71) Applicant: BASF SE, Ludwigshafen am Rhein (DE)

(72) Inventors: Claus TEUBER, Ludwigshafen am Rhein (DE); James H. EHLERS, Florham Park, NJ (US); Olaf HUBER, Ludwigshafen am Rhein (DE); Omar Osama Mohamed Mohamed ELBADRAWI, Madrid (ES); Jan SCHOENEBOOM, Ludwigshafen am Rhein (DE); Christopher Alec ANDERLOHR, Ludwigshafen am Rhein (DE); William Alexander MARVIN, Bridgewater, NJ (US); Steffen KLOSTERHALFEN, Ludwigshafen am Rhein (DE); Martin BINDER, Ludwigshafen am Rhein (DE); Bastian GRUMBRECHT, Ludwigshafen am Rhein (DE)

(21) Appl. No.: 18/854,262

(22) PCT Filed: Apr. 4, 2023
(86) PCT No.: PCT/EP2023/058884

§ 371 Date: Oct. 4, 2024

(30) Foreign Application Priority Data
Apr. 4, 2022 (EP) 22382320.4

(51) Int. Cl. G16C 20/10; G06Q 50/04

(52) U.S. Cl. CPC G16C 20/10; G06Q 50/04

(57) ABSTRACT

The present disclosure relates to product carbon footprints, and particularly to system and a computer-implemented method for monitoring and/or controlling production of products using a production process in which an allocation rule is applied for allocating emissions contributing to the product carbon footprint, PCF, of the products among at least two different products. The method comprises receiving production process data comprising information about at least one process step producing at least two output materials within the production process; receiving a first allocation rule and a second allocation rule different to the first allocation rule; determining, based on the production process data, at least two products affected by the first allocation rule; determining, for the affected products, a first PCF while applying the first allocation rule; determining, for the affected products, a second PCF while applying the second allocation rule; determining, based on a comparison of the first PCF and second PCF with each other, an operational instruction; and outputting the determined operational instruction.

The present invention relates to a method for monitoring an environmental impact of a product. In particular the present invention relates to a system and a computer-implemented method for monitoring and/or controlling production of products using a production process in which an allocation rule is applied for allocating emissions, e.g. greenhouse gas emissions, contributing to the product carbon footprint, PCF, of the products among at least two different products. Further, the invention relates to the use of the result of such a method for monitoring and/or controlling production of products, and a computer-readable medium.

The significance of climate protection measures is growing rapidly in the perception of the public, regulators and financial investors. Major companies have announced ambitious short-term CO2 reduction targets, including emissions related to purchased raw materials as, for example, required by the Science-Based Targets Initiative (SBTI). Therefore, transparency on product carbon footprints (PCF) and options to reduce the PCF are increasingly demanded.

PCFS are a measure to determine the amount of greenhouse gas emission caused to produce the respective product. PCFs are an important means to achieve a reduction in greenhouse gas emissions if those products with the lowest PCF are chosen for consumption or for further processing downstream in the value chain. For this purpose, it is of high importance that the reported PCF of any product is as accurate as possible.

An environmental impact may be measured by the PCF of the product. PCFs are often calculated by computer programs receiving the required input and subjecting them to an algorithm which calculates the PCF therewith. Thereby, for a linear production chain, i.e., a raw material is processed in multiple processing steps into one single product, this calculation is a straightforward addition of the contributions. However, the calculation becomes more difficult if a processing step has more than one output used to produce multiple products. An example from a chemical plant is the reaction of ethylene oxide with ammonia to form three reaction products: monoethanolamine, diethanolamine and triethanolamine. Each of these reaction products is used to produce products in separate further processing steps. The PCF calculation algorithm has to distribute the greenhouse emissions contributing to the PCF of the products for this reaction among the three products. This distribution is called allocation of the emissions contributing to the PCF of the products.

There may, therefore, be a need for providing improved means for controlling a production process to which at least one allocation of emissions contributing to a product's PCF is applied. The object is solved by the subject matter of the independent claims, wherein further embodiments are incorporated in the dependent claims.

In a first aspect, there is provided a computer-implemented method for monitoring and/or controlling production of products using a production process in which an allocation rule is applied for allocating emissions, e.g. greenhouse gas emissions, contributing to the product carbon footprint, PCF, of the products among at least two different products. The method may be carried out by a suitable system comprising at least one computing device, and may be applied to the production process for producing at least two products, in e.g. a production plant, production network, or the like.

The method comprises the steps of:
Receiving production process data comprising information about at least one process step producing at least two output materials within the production process.
Receiving a first allocation rule and a second allocation rule different to the first allocation rule.
Determining, based on the production process data, at least two products affected by the first allocation rule.
Determining, for the affected products, a first PCF while applying the first allocation rule.
Determining, for the affected products, a second PCF while applying the second allocation rule.
Determining, based on a comparison of the first PCF and second PCF with each other, an operational instruction.
Outputting the determined operational instruction.

In this way, the production of the products, in e.g. a production plant, can at least be monitored, but particularly controlled, taking into account the PCF of the product or products to be produced with the production process and the allocation rule applied thereto. In particular, the operational instruction can be used to monitor and/or control the production process, for example in order to monitor the PCF of the product(s), to control the production to achieve a certain, e.g. predetermined, PCF for the product(s), which can be influenced by the allocation rule applied respectively, etc. For example, the method allows to determine, e.g. calculate, the PCF of even at least two products, which are produced by the process step having more than one output used to produce the at least two products.

That is, the method described herein allows to monitor and/or control the production in terms of the PCF achieved or to be expected for the products even within a complex production process and/or a complex production environment producing at least two products using one or more allocation rules for allocating the emissions, e.g. greenhouse emissions, among the at least two products, wherein this is a complex, non-straightforward challenge in such a complex production process. Further, the method described herein can be used in many ways for and/or during the production of the product. For example, it can be ensured that the PCF of the products, preferably of all the products, at least approaches or matches a target PCF, or is documented for the product in terms of being tied thereto. Further, based on the knowledge of the allocation rule, controlling measures and/or interventions in the production process can be initiated to maintain or not exceed a target PCF, etc. Furthermore, with the knowledge about the allocation rule, it may be possible to adjust the production in such a way that the PCF value(s) decrease and are reliably low. Furthermore, the method can be used to ensure that the calculated PCF values also correctly reflect the actual conditions, e.g. by comparing the results with target or reference data, such as tabulated, industry-standard values). Noticeable deviations from such target or reference data can also be detected, which can then be analyzed and evaluated by an expert, etc.

In other words, the method, which may be implemented by a computer program including computer instructions which, when executed by a data processor or other computing device, is configured to determine the impact of the allocation rule applied in a particular process step within a complex production process and/or complex production environment on the PCF of the product(s) to be produced. This is achieved by receiving, by the computer program, information, e.g. a selection of the at least one process step of interest or under consideration, i.e. production process data for the at least one process step. In at least some embodiments, this may be one and in at least some other embodiments, more than one process step. The computer program determines the products which are produced with at least one output of the at least one process step directly affected by the allocation rule. Further, the computer program then calculates the PCF for the affected products using one allocation rule. For the calculation, according to at least some embodiments, the computer program may take into account the PCF of the raw materials and the energy usage of the at least one process step and/or each process step if more than one process steps are considered. This information may be provided by a data input, e.g. by the production process data. Then, the computer program calculates the PCF for said products using the same information about raw materials and energy usage, but using a different allocation rule. From a comparison of the PCFs determined by using the different allocation rules with each other, the computer programs determines the operational instructions and provides this for further processing. Thus, the method described herein allows to monitor and/or control the production process at least with respect to the effect or impact of the respective applied allocation rule and/or a changed allocation rule to or on the PCF of the product(s).

In the context of the present disclosure, the expression “monitoring the production” may be understood broadly, and may refer, for example, to any kind of monitoring of the production process in terms of tracking the product's PCF, validation and/or evaluation of the PCF, ensuring that the product's PCF reaches or not exceeds a target PCF, or the like, by monitoring the production process. Further, “monitoring the production” may also include planning the production, which can be done in a planning phase before implementing the actual production.

Further, the expression “controlling the production” may refer to any controlling measure or intervention within production, a production network, a production step, or the like, that affects the PCF of the product. This may comprise generating a control signal that modifies data within production, e.g. in or via a production control system, an enterprise resource planning systems, or the like. For example, such a controlling measure or intervention may, for example, comprise controlling the production in terms of the PCF of the raw material, e.g. by changing the raw material, a supplier of it, etc., of a process step, e.g. by changing an energy used for the process step, by technical modification of the production step by an modified physical or chemical influence on an input material of the production step, etc. In other words, controlling the production may change one or more production process parameters in the production reality and thus directly or indirectly control the PCF of the product. Further, “controlling the production” may also include planning the production, which can be done in a planning phase before implementing the actual production.

However, it is also possible that the “monitoring and/or controlling the production” comprises creating or modifying product information, such as the PCF of the product. For example, it is possible that if it is determined that the influencing factor changes the PCF of the product, the product information, such as the PCF of the product, is modified accordingly. The latter can be done via modifying a corresponding information, e.g. a dataset, in an enterprise resource planning (ERP) system, which manages the corresponding product information and assigns it to the product in a traceable way.

As used herein, the “production process data” may generally describe the production process of at least one input material, via typically a process step in which the input material is used or processed, or in which the input material is acted upon, to obtain an output material. It should be noted that in the context of the present disclosure, those production processes are considered from which at least two products result in order to apply the allocation rule accordingly. For example, the production process data may comprise one or more of process data comprising information about the process step(s) from the required raw material, which may also referred to as the input material of the at least one process step under consideration, to the product and/or the output material of the at least one process step. In at least some embodiments, the production process data may further comprise a carbon footprint of each raw material and energy data comprising information about the energy consumption for the at least one process step and/or each process step under consideration. Further, the process data may be gathered from the production plant. It can be gathered through an interface from a local or a remote database, or any other suitable data source. Preferably, the production process data is gathered through an interface to the ERP system. In this way, the production process data may be gathered from the ERP system. The ERP system may obtain the information from the production plant. In this case, the process data is gathered from the production plant via an ERP system. In this way, the process data is instantly updated once any change in the production plant or its surrounding occurs. Depending on the ERP system “instantly” typically means in less than or equal to one day, preferably less than or equal to six hours, in particular less than or equal to one hour. A typical example of such a change would be that the production plant receives insufficient reagent from a different factory and has to use an external supply instead. Such an external supply usually has a different product carbon footprint than the internal intermediate, hence changing the carbon footprint of the product produced in the production plant. Another advantage of an ERP is system is that the data is standardized and validated, i.e. it is reliable and typically does not need further validation. For example, the “production process data” may be received via an interface, e.g. a data interface, communication interface, etc.

The production process data may comprise information about which by-products are obtained in which amount. Some process steps may not produce any by-products, such as the assembly of steel parts. In this case, the production process data does not comprise information about by-products. However, many process steps produce by-products. A “by-product” in the context of the present disclosure refers to any good which is unavoidably obtained in a process step but cannot be used in a different process step. Sometimes, a by-product can be recycled, i.e. be subjected to another process step or multiple process steps to obtain a raw material or an intermediate which can be used as reagent in a process step. However, in some cases, there is no economically feasible use for the by-product. In this case, the by-product has to be disposed. It can, for example, be burned in an incineration. If the incineration is part of the production plant, the thermal and/or electrical energy regained has to be taken into account.

The production process data may comprise information about which intermediate or intermediates are obtained in each process step and at which yield. The “yield” in the context of the present invention refers to the percentage of outcome from a particular process step relative to the theoretical maximum. If the yield is 100%, for example if ingredients are mixed into a formulation, the production process data does not have to comprise information about the yield. However, the yield can be below 100% if there are losses in a process step. In chemical reactions, the yield is typically below 100%, because of side reactions and losses upon purifications. In other processes, yields can also be below 100%, for example if steel parts are cut or drilled, the chips may cause a loss unless they can be reused.

An “ERP system” in the context of the present disclosure shall have its common meaning. A typical ERP system provides an integrated and continuously updated view of core business processes using common databases maintained by a database management system. ERP systems typically track business resources such as cash, raw materials, production capacity and the status of business commitments: orders, purchase orders, and payroll. The applications that make up the system typically share data across various departments such as those responsible for manufacturing, purchasing, sales, accounting, that provide the data.

The term “allocation rule” may be understood as any rule that determines the distribution of emissions among individual products and/or can control this distribution, e.g. by intervening in production. For example, the allocation rule may be understood as a rule, an instruction, or the like, specifying how the emissions affecting the PCF, e.g. greenhouse emissions, generated at least in the process step under consideration are to be distributed among the products to be produced, which may be produced in one or more further process steps downstream to the process step under consideration. In an example an allocation rule may be a rule specifying how the emissions affecting the PCF, e.g. greenhouse emissions, generated at least in the process step under consideration are to be distributed among the respective processes involved in the production of a product.

The distribution of emissions among the products and/or processes may, for example, be done according to their mass, volume, number of units, moles (in the case of chemical reactions), or other quantifiable units. An example from a chemical plant is the reaction of ethylene oxide with ammonia to form three reaction products: monoethanolamine, diethanolamine and triethanolamine. Each of these reaction products is used to produce products in separate further processing steps. The PCF calculation algorithm has to distribute the carbon dioxide emitted for this reaction among the three products and/or corresponding process steps. This distribution is called allocation and may be expressed in a corresponding, preferably computer-readable, allocation rule. As used herein, the “allocation rule” may be received via an interface, e.g. a data interface, communication interface, etc.

The methods steps directed to the determination, e.g. calculation, of the products affected by the allocation rule, the first and/or second PCF, and/or the operational instruction may be performed by e.g. a suitable data processor or other computing device, which may be operatively connected to the input interface via which the production process data is received. Outputting the operational instruction may be performed by e.g. a suitable output interface, e.g. a data interface, communication interface, etc.

As used herein, the “operational instruction” may be understood broadly, and may refer to any information about the production process or any information which triggers or can be used to trigger an action related to the production. In a simple case, the operational instruction may be used to create or adapt the product information, e.g. the PCF of the product or products. In another simple case, it may be used to evaluate and/or verify the PCF of the product or products, taking into account the allocation rule applied. However, it may also be possible that the operational instruction comprises one or more computer instructions for controlling, modifying, etc., the production process, for example via a production control system, the ERP system, or the like.

As used herein, “outputting” the at least one influencing factor may be understood as writing the carbon footprint on a non-transitory data storage medium, display it on a user interface or both. It is also possible to provide the output through an interface to a customer, for example to the customers supply chain system, ERP system or the like. It is also possible to provide the output through an interface to the ERP system of the producer itself from where it can be distributed to where this information is needed.

When the operational instruction is output onto a user interface, the user interface preferably uses graph technology. In this way, it is possible to analyze the contributions along the production process in order to monitor/and or control the production process and thereby minimize the carbon footprint for the products. It is also possible to monitor and/or control changes of the carbon footprint upon changes in the production process. In addition, the output can be used to simulate effects of changes, for example by manually changing certain values and see its effect on the carbon footprint of the product. For example, the effect of replacing a particular raw material by one having lower carbon footprint for each product may be analyzed.

Further, as used herein, the term “carbon footprint” may be understood as a total amount of greenhouse gases emitted or removed in the whole process from extracting natural resources to the product as it leaves a production plant. In the context of the present disclosure, the carbon footprint does not include any greenhouse gas emission later on in the lifetime of a product. For example, for a car, the carbon footprint in the context of the present disclosure is the amount of greenhouse gases emitted to produce the car, but not the emissions caused by using the car once it has left the production plant. The amount of the carbon footprint is typically expressed as carbon dioxide equivalents, so the amount of carbon dioxide with the same effect on global climate as the actually emit-ted greenhouse gases.

Greenhouse emissions and/or gases may comprise carbon dioxide, carbon monoxide, nitrous oxide, methane, ozone, chlorofluorocarbons, hydrofluorocarbons. These can be translated into carbon dioxide equivalents according to IPCC 5th assessment report (cf. standards such as ISO 14067 for carbon footprint of products or the Greenhouse Gas Protocol Product Standard WRI & WBCSD, 2011).

The method described herein can be applied to a wide variety of products which are produced from raw materials. The term “product” as used herein, generally refers to any good which can be sold to others at any point in the value chain. This may include final products for end consumers, for example cars, paints, toys or medicaments; this may also include goods which are typically sold to other companies which further process them, for example steel parts for machines, plastic pellets for extrusion or chemical compounds, for example acrylic acid to produce superabsorbers for diapers; this may also include goods very early in the value chain like crude oil fractions, for example naphtha, agricultural products, for example soy beans, or purified sand for glass production.

The term “raw material” as used in the present disclosure refers to any good which is bought from suppliers and brought to the production plant. A raw material can be on any step along the value chain like the product described above. This means, the product of the one production plant can be the raw material of the other production plant. Raw material can also include very fundamental goods like air, water, natural gas or salt.

An “intermediate” refers to a good, such as a substance, which is neither a raw material nor a product, but is made from raw materials or earlier intermediated and is processed further into other intermediates and finally into the product. The intermediate may be associated with a corresponding process step in which it is produced, used, transported, etc.

A “production plant” as used in the present disclosure is any facility which is able to produce any kind of good which is sold to an end customer or further processed in a different production plant. A production plant can be on one single site or on multiple. If the production plant is in multiple sites, these have to be under common control which is typically the case if they belong to the same company or to affiliated companies. Examples for plants are power plants, steel manufacturing plants, oil producing plants, oil refineries, chemical plants, plants for manufacturing pharmaceuticals, plants for manufacturing construction materials, machine manufacturing plants, automobile manufacturing plants, plants for manufacturing textiles, plants for manufacturing furniture, food production plants, plants for manufacturing consumer electronics such as cell phones, plants for manufacturing and/or processing of paper, such as a printing press.

A “process step” as used herein may be understood as a series of acts onto the raw material(s) which cannot be reasonably separated in time or space. Typically, all acts of one process step take place in one building using a certain dedicated equipment.

The method according to the present invention is particularly useful for production plants which execute interconnected process steps. The term “interconnected” in the context of the present in invention means that at least one process step uses two intermediates of different other process steps or uses one intermediate of different other process steps each producing this intermediate or yields two intermediates which are used in two different other process steps. Hence, preferably, the production plant executes interconnected process steps. Even more preferably, the production plant is a chemical production plant executing interconnected process steps. Often, the interconnected process steps are executed in different factories, maybe on different sites, potentially operated by different group companies.

According to an embodiment, the method may be carried out prior to an actual change from the first to the second allocation rule within a production planning phase. For example, the determined operational instruction may first be used only to estimate or predict the effect of the particular allocation rule on the PCF of the product or products before the allocation rule is actually changed or used as a replacement for a previously used or planned allocation rule. In this way, production or its control can be checked for the impact of the allocation rule without immediate impact on the actual production process and/or the PCF of the product(s).

In an embodiment, different first and/or second allocation rules may be received and/or applied for different process steps. In other words, the at least one process step considered can be selected from several different process steps provided for the production process, e.g. also interrelated process steps, wherein different allocation rules are or will be specified for different process steps. That is, the method described herein may also be applied to complex production processes or complex production environments to which more than one process step and/or more than one allocation rule is to be applied.

According to an embodiment, there may be specified, e.g. predetermined, a target PCF for at least one of the at least two different products, and the allocation rule suitable for achieving the target PCF may be determined based on the determined operational instruction. In other words, a target PCF can be specified for a product and the allocation rule appropriate or expected to achieve the target PCF may be determined based on the determination of the operation instruction and/or one or more of the determining, e.g. calculation, steps involved as described above. This allows production to be controlled in such a way that a specified PCF is actually at least approximately achieved.

In an embodiment, the at least one process step may be associated with multiple allocation rules, and the method may further comprise comparing the second PCF to a PCF reference value associated with the product, and determining a specific allocation rule among the multiple allocation rules that causes the PCF of the product to come closest to the PCF reference value. In other words, the method, e.g. the computer program, may be configured to estimate which allocation rule comes closest to reality, e.g. by comparing at least the second PCF to the PCF reference value, e.g. to a market's standard or the like. If the computer program has these as an input, it may be able to output, e.g. display, the deviations. These may be analyzed, and it may be determined if differences in production methods are the reason for the deviation. If not, the allocation rule which comes closest to the reference may be selected as the most appropriate, suitable or best one. For example, the PCF reference value may, for example, be a PCF standardized for the product or a product group contained therein or a PCF customary in the market, e.g. also a PCF of a competitor's product, a PCF specified in some other way, or of the like. This allows the PCF of the product(s) to be monitored and/or controlled even more accurately, or the production to be monitored and/or controlled taking into account the further reference value.

According to an embodiment, the at least one process step may be associated with multiple allocation rules, and the method may further comprise: determining, based on the production process data, the products affected by the multiple allocation rules, determining, for the affected products, a first PCF while applying an individual one of the multiple allocation rules, determining, for the affected products, a second PCF while applying the individual one of the multiple allocation rules, and determining, based on a comparison of the first PCF and second PCF with each other, for each determination applying the multiple allocation rules, the operational instruction. In other words, if the at least one process step under consideration includes multiple allocation rules, the determination of the operational instruction may be performed for each individual allocation rule. In this way, even complex production processes or production environments can be monitored and/or controlled with regard to their influence by the multiple allocation rules.

In an embodiment, the method may further comprise applying an optimization algorithm, e.g. a solver or the like, utilizing e.g. a cost function or any other suitable optimization means to determine at least one allocation rule to be changed or replaced within the at least one process step and/or within the production process by another allocation rule that is determined by the optimization algorithm to adjust, e.g. decrease or minimize, the PCF. This allows a particularly precise selection of a suitable allocation rule.

According to an embodiment, the operational instruction may be output via a user interface. The user interface may comprise a graphical user interface configured to represent the operational instruction in a comprehensible context with the production process, the at least one process step, the PCF of the product(s) and/or the monitoring and/or controlling of the production process. For example, the user interface may output, e.g. display, the absolute or the relative change for each such product. Hence, the impact of the allocation rule on the PCF for the products can be visualized and/or evaluated.

In an embodiment, information about the second allocation rule and/or the second allocation rule to be applied for determining the second PCF is received via a user interface configured to allow changing at least the allocation rule of the at least one process step. For example, the user interface may comprise a graphical user interface (GUI), via which a user, e.g. an operator of the production process, can at least select the second allocation rule. It may also allow to modify an existing allocation rule, e.g. the first allocation rule, into the second allocation rule which differs in at least one parameter from another allocation rule, e.g. the first allocation rule. Further, in at least some embodiments, the at least one process step under consideration may be selected and/or configured via the user interface. This allows a wide monitoring and/or wide control of the production process.

According to an embodiment, the method may further comprise replacing the first allocation rule by the second allocation rule if the determined operational instruction meets a replacement criterion. For example, the second allocation rule may be the allocation rule determined to be most appropriate. It can also be determined by the optimization algorithm described above. That is, the replacement criterion may be the result of e.g. the optimization algorithm, or the like. In this way, production can be controlled with regard to the allocation rule to be used and/or the PCF of the product(s) to be achieved by active intervention in the control system by means of the replaceable allocation rule. The allocation rule can be used at any suitable point of the production control, for example in the ERP system or the like.

In an embodiment, the first PCF and/or the second PCF may be determined by receiving a carbon footprint of raw material used in the at least one process as input material, receiving energy data comprising information about an energy consumption for the at least one process step, and determining the first PCF and/or the second PCF of the product taking into account the production process data, the carbon footprint of raw material and/or the energy data.

For example, calculating the PFC of the product or the intermediate comprises summing the carbon footprints of each raw material used in a particular process step as contained in the production process data. If a process step requires an intermediate from a different process step, the sum of the carbon footprint of the raw material for this earlier process step is determined and used as input for the later process step. It may be necessary to repeat this if the earlier process step again uses an intermediate of an even earlier process step. If one process step yields more than one intermediate, for example two or three, it is necessary to share the carbon footprint of the raw materials among these intermediates. The share for each intermediate should reflect the raw material us-age for each intermediate. In some cases, two intermediates are formed at the same amount, so the carbon footprint of the raw materials can be equally shared among them. In other cases, significantly more of one intermediate is formed than the other, for example 90% of intermediate 1 and 10% of intermediate 2. The carbon footprint should be shared accordingly. Hence, preferably, in the method of the present invention determining the carbon footprint involves calculating the carbon footprint for an intermediate produced in a preceding process step and using the carbon footprint of the intermediate as input for the calculation of the carbon footprint of a subsequent process step. In particular, in interconnected production processes, the calculation of the carbon footprint can be facilitated by subdividing it into analogous calculation parts, one for each process step.

The production process data may comprise information about any direct greenhouse gas emissions by the process step. Such direct greenhouse gas emissions often stem from a chemical reaction of the raw materials which either contain greenhouse gases or generate greenhouse gases during the process step, for example by heating. A typical example is cement production in which carbon dioxide evolves from heating the raw materials, in particular from heating limestone. The information about direct greenhouse gas emissions usually contains the information which green-house gas is emitted at which amount. The amount can be given relative to the amount of raw materials or relative to the amount of product or intermediate of the respective process step. The latter can be derived from the former by multiplying with the yield of the process step.

In the easiest case, one or multiple raw materials may be processed in one process step to arrive at at least two products. For example, a given cable as raw material could be cut into different lengths to obtain two cables of different lengths as different products. In most cases, however, the production processes are more complicated. Multiple raw materials are processed into various intermediates which are processed into various products, wherein one raw material can be used to produce more than one intermediate and one intermediate may be used to pro-duce more than one product. In such a situation, the final carbon footprint of one product become dependent on the amount of other products produced at the production plant. Hence, typically the production process data comprise the information which reagents are required at which amounts for each process step for all products having at least one reagent or intermediate in common. For many production plants, the production process data comprise the information which reagents are required at which amounts for each process step for at least two products having at least one reagent or intermediate in common. For complex production plants the production process data comprise the information which reagents are required at which amounts for each process step for at least five or at least ten products having at least one reagent or intermediate in common.

The production process data is typically obtained, received, etc. through an interface, e.g. a data interface, communication interface, etc. The production process data may be obtained from e.g. a production plant. It can be obtained through an interface to a local or a remote database. Preferably, the production process data is obtained through an interface to an enterprise resource planning (ERP) system. In this way, the production process data may be obtained from the ERP system. The ERP system may obtain the information from the production plant. In this case, the production process data is obtained from the production plant via an ERP system. In this way, the production process data is instantly updated once any change in the production plant or its surrounding occurs. Depending on the ERP system “instantly” typically means in less than or equal to one day, preferably less than or equal to six hours, in particular less than or equal to one hour. A typical example of such a change would be that the production plant receives insufficient reagent from a different factory and has to use an external supply instead. Such an external supply usually has a different product carbon footprint than the internal intermediate, hence changing the carbon footprint of the product produced in the production plant. Another advantage of an ERP is system is that the data is standardized and validated, i.e. it is reliable and typically does not need further validation.

A second aspect relates to the use of an operational instruction, determined by the method of the first aspect, in monitoring and/or controlling a production process. Possible monitoring and/or controlling applications are described above with respect to the first aspect, and reference is made to the description above. For example, using the determined operational instruction allows to monitor and/or control the production in terms of the PCF achieved or to be expected for the products even within a complex production process and/or a complex production environment producing at least two products using one or more allocation rules for allocating the emissions, e.g. greenhouse emissions, among the at least two products, wherein this is a complex, non-straightforward challenge in such a complex production process.

In a third aspect, there is provided a non-transitory computer readable data medium storing a computer program including instructions for executing steps of the method according to the first aspect. Computer readable data medium include hard drives, for example on a server, USB storage device, CD, DVD or Blue-ray discs. The computer program may contain all functionalities and data required for execution of the method according to the first aspect, or it may provide interfaces to have parts of the method processed on remote systems, for example on a cloud system.

According to a fourth aspect, there is provided a system for monitoring and/or controlling production of products using a production process, in which production process an allocation rule is applied for allocating emissions contributing to the product carbon footprint (PCF) of the products among at least two different products. The system may be configured to monitor and/or control the production process by data exchange with a production control system, an ERP system or the like, by analysis, adaptation and/or output of production information, and/or by active intervention in the production control system, in the ERP system, etc. Unless explicitly described differently hereafter, the description above relating to the method including preferred embodiments also applies to the system. The system can be a computing device, for example, a computer, tablet, or smartphone, or any other suitable computing device. In at least some embodiments, the computing device may have a communication interface and/or network connection in order to communicate with other computing devices, such as servers or a cloud network.

The system comprises an input interface that is configured to receive production process data comprising information about at least one process step producing at least two output materials within the production process, and a first allocation rule and a second allocation rule different to the first allocation rule. The system further comprises a data processor that is configured to determine, based on the production process data, at least two products affected by the first allocation rule and/or second allocation rule, determine, for the affected products, a first PCF while applying the first allocation rule, determine, for the affected products, a second PCF while applying the second allocation rule, determine, based on a comparison of the first PCF and second PCF with each other, an operational instruction. The system further comprises an output interface, configured to output the determined an operational instruction for further processing, e.g. for its evaluation, for displaying, for controlling the production process, etc.

According to an embodiment, the output interface may comprise a user interface configured to display the determined operational instruction. The user interface is preferably configured to display the influencing factor and/or carbon footprint of the product and each contribution, preferably comprising the contribution of the raw materials, the contribution of the energy, and the contribution of the direct emissions of each process step. Preferably, the user interface uses graph technology. The user interface may provide an overview of each process step, its raw materials and energy required, the connection with other process steps. The user interface may also provide the carbon footprint for each process step, in particular it may display the carbon footprint originating from the raw materials, from the energy consumption, and from the direct greenhouse gas emissions separately and in aggregated form.

Preferably, the system is adapted to receive updated data at any time and can update the output in real time, which usually means within less than a few minutes, preferably within less than a minute, for example within 1 to 30 seconds.

It is noted that embodiments of the invention are described with reference to different subject-matters. In particular, some embodiments are described with reference to method-type claims whereas other embodiments are described with reference to apparatus or device-type or system-type claims. However, a person skilled in the art will gather from the above and the following description that, unless otherwise notified, in addition to any combination of features belonging to one type of subject-matter also any combination between features relating to different subject-matter is considered to be disclosed with this application. Further, all features can be combined providing synergetic effects that are more than the simple summation of the features.

These and other aspects of the present disclosure will become apparent from and elucidated with reference to the embodiments described hereinafter.

Exemplary embodiments of the invention will be described in the following with reference to the following drawings.

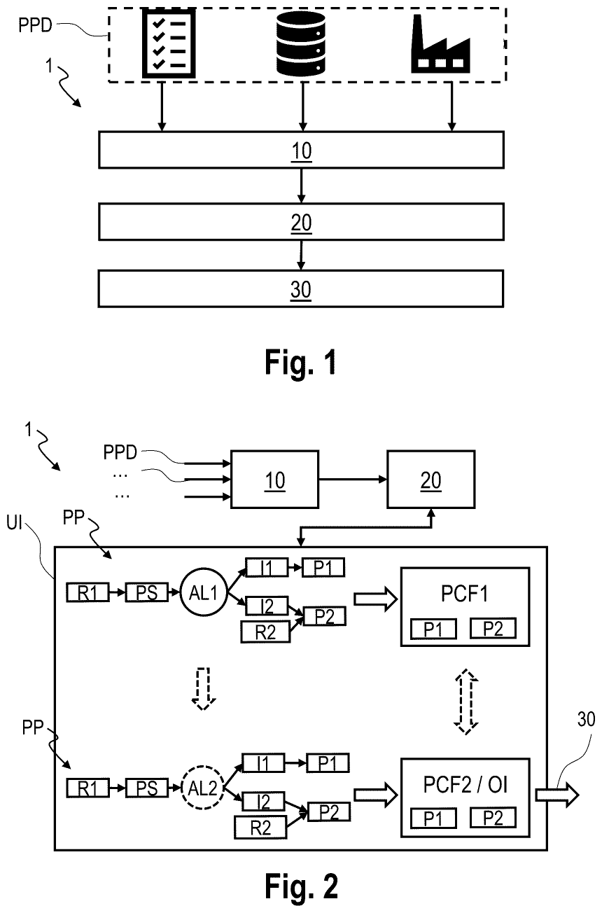

FIG. 1 illustrates in a schematic block diagram a system for controlling production of products using a production process according to the present disclosure.

FIG. 2 illustrates in a schematic block diagram a system and principle for controlling production of products using a production process according to the present disclosure.

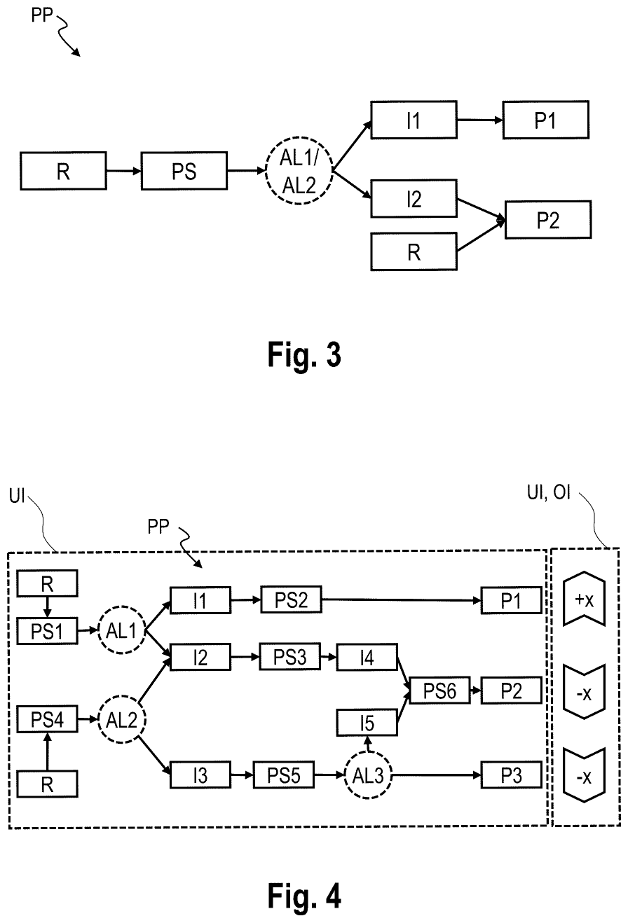

FIG. 3 illustrates in a schematic block or process diagram an example of a production process or production chain in which an allocation rule is to be applied for allocating emissions contributing to the product carbon footprint (PCF) of the product(s) among at least two different products, to which exemplary production process or production chain a method or system for controlling production of products using a production process according to the present disclosure can be applied.

FIG. 4 illustrates in a schematic block or process diagram an example of a production process or production chain in which an allocation rule is to be applied for allocating emissions contributing to the product carbon footprint (PCF) of the product(s) among at least two different products, to which exemplary production process or production chain a method or system for controlling production of products using a production process according to the present disclosure can be applied.

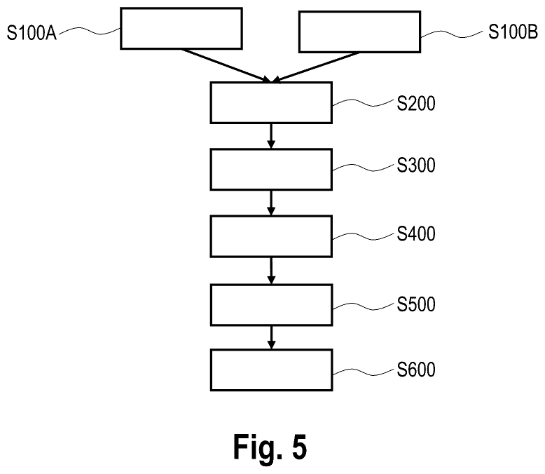

FIG. 5 illustrates in a flow chart a computer-implemented method for controlling production of products using a production process according to the present disclosure.

The drawings are merely schematic representations and serve only to illustrate the invention. Identical or equivalent elements are consistently provided with the same reference signs.

FIG. 1 illustrates in a schematic block diagram a system 1 configured to monitor and/or control production of products using a production process, which may also be referred to as production chain, in which production process an allocation rule AL1, AL2, AL3 (see e.g. FIG. 2, FIG. 3 or FIG. 4) is applied for allocating emissions contributing to the product carbon footprint (PCF), of the products among at least two different products. The system 1 is any suitable computing device and comprises an input interface 10, a data processor 20, and an output interface 30. The processor 20 is operatively connected to each one of the input interface 10 and the output interface 30.

The input interface 10 is e.g. a data interface, communication interface, or the like, configured to receive production process data PPD comprising at least information about at least one process step PS (see e.g. FIG. 3, FIG. 2 or FIG. 3) producing at least two output materials within the production process, and a first allocation rule AL1, AL2, AL3 (see e.g. FIG. 3, FIG. 2 or FIG. 3) and a second allocation rule AL1, AL2, AL3 (see e.g. FIG. 3, FIG. 2 or FIG. 3) different to the first allocation rule AL1, AL2, AL3. For this purpose, the input interface 10 is operatively connected to, for example, one or more suitable data sources, such as an enterprise resource planning system (ERF), a supplier database, or the like, which may collect and/or provide the process data PPD. For example, the process data PPD may comprise one or more of process data comprising information about the process steps from the required raw materials to the product, a carbon footprint of each raw material, and energy data comprising information about the energy consumption for each process step. The first and second point in time may be two different points in time within the production process, different links in the production chains, or the like.

The processor 20 is configured, e.g. by executing computer instructions of a respective computer program, to determine, e.g. calculate, based on the production process data PPD, at least two products P1, P2, P3 (see e.g. FIG. 2, FIG. 3 or FIG. 4) affected by the first allocation rule AL1, AL2, AL3. The processor 20 is further configured to determine, e.g. calculate, for the affected products P1, P2, P3, a first PCF PCF1 (see e.g. FIG. 2, FIG. 3 or FIG. 4) while applying the first allocation rule AL1, AL2, AL3. Further, the processor 20 is configured to determine, e.g. calculate, for the affected products P1, P2, P3, a second PCF PCF2 (see e.g. FIG. 2, FIG. 3 or FIG. 4) while applying the second allocation rule AL1, AL2, AL3. The processor 20 is further configured to determine, e.g. calculate, based on a comparison of the first PCF PCF1 and second PCF PCF2 with each other, an operational instruction OI (see e.g. FIG. 2, FIG. 3 or FIG. 4). For example, the operational instruction may relate to or may comprises any information, measure, or the like, by which the production process and/or the PCF of the product(s) can be monitored and/or controlled and/or the corresponding product information can be created and/or amended. In a simple case, the operational instruction may be used to create or adapt the product information, e.g. the PCF of the product or products. In another simple case, it may be used to evaluate and/or verify the PCF of the product or products, taking into account the allocation rule applied. However, it may also be possible that the operational instruction comprises one or more computer instructions for controlling, modifying, etc., the production process, for example via a production control system, the ERP system, or the like.

The output interface 30 is any suitable data interface, communication interface or the like, configured to output the operational instruction OI for further processing within the production process and/or production environment. For example, the output interface 30 The output interface 30 may, for example, comprise or may be operatively connected to a user interface UI (see e.g. FIG. 2 or FIG. 4) for displaying the at least operational instruction OI. However, it may also be operatively connected to a production control system (not shown), the ERP (not shown) or the like, so that the system 1 may monitor and, particularly computationally, control the production based on the at least one operational instruction OI, thereby controlling the production process taking into account the allocation rule applied or to be applied and/or the PCF of the product(s). The control of the production process may comprise, for example, controlling one or more parameters of the production process, such as raw material, energy consumption, etc., thereby also controlling the PCF of the product P1, P2, P3 accordingly.

It is noted that the processor 20 and/or output interface 30 may be operatively connected to a production control system, the ERP, or the like, and configured to output one or more control signals configured to control the production control system, the ERP, the supply chain, etc.

Optionally, the processor 20 is configured to determine the operational instruction OI prior to an actual change from the first to the second allocation rule AL1, AL2, AL3 within a production planning phase. For example, the processor may be configured to first only estimate determined the effect of the particular allocation rule AL1, AL2, AL3 or its change on the PCF of the product or products P1, P2, P3 before the allocation rule AL1, AL2, AL3 is actually applied or changed.

Further optionally, the processor 20 is configured to receive and/or apply different first and/or second allocation rules AL1, AL2, AL3 for different process steps PS1-PS6 (see e.g. FIG. 2, FIG. 3 or FIG. 4). In other words, the at least one process step SP under consideration can be selected from several different process steps PS1-PS6 provided for the production process, e.g. also interrelated process steps, wherein different allocation rules AL1, AL2, AL3 are specified for at least some of the different process steps PS1-PS6.

Optionally, the processor 20 is configured to determine target PCF that may be specified, e.g. predetermined, for at least one of the at least two different products, and to determine the allocation rule AL1, AL2, AL3 suitable, or even best suitable, for achieving the target PCF based on the determined operational instruction OI.

Further optionally, the at least one process step PS1-PS6 is associated with multiple allocation rules AL1, AL2, AL3, and the processor 20 is further configured to compare at least the second PCF PCF2 to a PCF reference value associated with the product P1, P2, P3, and to determine a specific allocation rule among the multiple allocation rules AL1, AL2, AL3 that causes or is expected to cause the PCF of the product to come closest to the PCF reference value.

Optionally, the at least one process step PS1-PS6 is associated with multiple allocation rules AL1, AL2, AL4, and the processor 20 is further configured to determine, based on the received production process data PPD, the products P1, P2, P3 affected by the multiple allocation rules, to determine, for the affected products P1, P2, P3, optionally for each of the affected products P1, P2, P3, the first PCF PCF1 while applying an individual one of the multiple allocation rules AL1, AL2, AL3, to determine, for the affected products P1, P2, P3, optionally for each of the affected products P1, P2, P3, the second PCF PCF2 while applying the or another individual one of the multiple allocation rules AL1, AL2, AL3, and to determine, based on a comparison of the first PCF and second PCF with each other, for each determination applying the multiple allocation rules AL1, AL2, AL3, the operational instruction OI.

Further optionally, the processor 20 is further configured to apply an optimization algorithm, e.g. a solver or the like, utilizing e.g. a cost function or any other suitable optimization means, to determine at least one allocation rule AL1, AL2, AL3 to be changed or replaced within the at least one process step PS1-PS6 and/or within the production process by another allocation rule AL1, AL2, AL3 that is determined by the optimization algorithm to adjust, e.g. decrease or minimize, the PCF of the product(s) P1, P2, P3.

Optionally, the processor 20 is further configured to replace one allocation rule AL1, AL2, AL3 by another allocation rule AL1, AL2, AL3 if the determined operational instruction OI meets a replacement criterion. For example, the replacing allocation rule may be the allocation rule determined to be most appropriate. It can also be determined by the optimization algorithm described above. By way of example, the processor 20 is configured to implement the operation instruction, or more specifically the another allocation rule AL1, AL2, AL3 within the production process

Further optionally, the processor 20 is further configured to determine the first PCF PCF1 and/or the second PCF PCF2 by receiving a carbon footprint of raw material used in the at least one process as input material, receiving energy data comprising information about an energy consumption for the at least one process step, e.g. via the input interface 10, and to determine the first PCF PCF1 and/or the second PCF PCF2 of the product P1, P2, P3 taking into account the production process data PPD, the carbon footprint of raw material and/or the energy data.

For example, calculating the PFC of the product or the intermediate comprises summing the carbon footprints of each raw material used in a particular process step as contained in the production process data. If a process step requires an intermediate from a different process step, the sum of the car-bon footprint of the raw material for this earlier process step is determined and used as input for the later process step. It may be necessary to repeat this if the earlier process step again uses an intermediate of an even earlier process step. If one process step yields more than one intermediate, for example two or three, it is necessary to share the carbon footprint of the raw materials among these intermediates. The share for each intermediate should reflect the raw material us-age for each intermediate. In some cases, two intermediates are formed at the same amount, so the carbon footprint of the raw materials can be equally shared among them. In other cases, significantly more of one intermediate is formed than the other, for example 90% of intermediate 1 and 10% of intermediate 2. The carbon footprint should be shared accordingly. Hence, preferably, in the method of the present invention determining the carbon footprint involves calculating the carbon footprint for an intermediate produced in a preceding process step and using the car-bon footprint of the intermediate as input for the calculation of the carbon footprint of a subsequent process step. In particular, in interconnected production processes, the calculation of the carbon footprint can be facilitated by subdividing it into analogous calculation parts, one for each process step.

FIG. 2 illustrates in a schematic block diagram the system 1 described above and the principle for controlling, via the input interface 10, the processor 20 and the output interface 30, wherein the production of products P1, P2, P3 starts from a raw material R1 using a production process PS via an intermediate I1, an intermediate I2, and a further raw material R2. In this example, the production process PP runs in the direction of the arrows connecting the individual process elements, wherein at least the two products P1, P2 or possibly more than two products P are produced at the end. As indicated in FIG. 2 by the designation UI, the user interface UI can be configured to output, e.g. display, the first PCF designated as PCF1 and the second PCF2 designated as PCF2, as well as the operational instruction OI. Further, as indicated in FIG. 2 by the arrow 30, the output interface 30 may also be connected to further processing means, such as the production control system, ERP system, or the like, in order to actively control the production process and/or to control the PCF of one or more of the products P1, P2. In FIG. 2, the dashed circle denoted AL indicates that the allocation rule can be changed to be processed by the processor 20. This can also be initiated via manipulation of the user interface UI by a user, e.g. an operator of the production system 1.

Optionally, the operational instruction OI is output via the user interface UI. The user interface UI may comprise a graphical user interface (GUI) configured to represent the operational instruction OI in a comprehensible context with the production process, the at least one process step PS, the raw materials R1, R2, the first and second PCF1, PCF2, the PCF of the product(s) P1, P2, P3, etc., and/or monitoring and/or controlling means for the production process. For example, the user interface UI may output, e.g. display, the absolute or the relative change of the PCF for each such product P1, P2, representing the operational instruction OI or a part of it.

Further optionally, the user interface UI is configured to select the allocation rule AL1, AL2 used to determine the second PCF2. For example, the user interface UI may comprise a graphical user interface (GUI), via which a user, e.g. an operator of the production process, can at least select the allocation rule for determining the second PCF2.

FIG. 3 illustrates in a schematic block or process diagram an example of a production process PP or production chain in which an allocation rule AL is to be applied for allocating emissions contributing to the product carbon footprint (PCF) of the product(s) among at least two different products P1, P2. Again, the production of products P1, P2, P3 starts from a raw material R1 using a production process PS via an intermediate I1, an intermediate I2, and a further raw material R2. In this example, the production process PP runs in the direction of the arrows connecting the individual process elements, wherein at least the two products P1, P2 or possibly more than two products P are produced at the end. Again, in FIG. 3, a dashed circle designated by AL1 indicates that this one allocation rule AL1 can be changed to another allocation rule AL2, wherein applying another allocation rule AL influences at least the allocation of greenhouse emissions contributing to the PCF of product P1 and/or P2 among the products P1, P2. By applying the system 1 or method(s) described herein to the example illustrated in FIG. 3, the allocation of the greenhouse emissions can be monitored and/or controlled, as described herein.

FIG. 4 illustrates in a schematic block or process diagram a further example of a production process PP or production chain in which, however, multiple allocation rules AL1, AL2, AL3 are to be applied for allocating greenhouse emissions contributing to the PCF of the product(s) among, here three, different products P1, P2, P3. It should be noted that the system 1 or the method described herein can also be applied to more complex production processes, e.g. comprising more than the illustrated number of raw materials R, intermediates I, process steps PS, by following the principle described herein. Again, in FIG. 4, dashed circles designated by AL1, AL2, AL3 indicate that this individual one allocation rule AL1, AL2, AL3 can be changed to another allocation rule AL, wherein applying another allocation rule AL influences at least the allocation of greenhouse emissions contributing to the PCF of product P1, P2 and/or P3 among the products P1, P2, P3. By applying the system 1 or method(s) described herein to the example illustrated in FIG. 4, the allocation of the greenhouse emissions can be monitored and/or controlled, as described herein. Further, FIG. 4 illustrates an exemplary user interface UI, which indicates an absolute or relative change in the PFC of the respective product P1, P2, P3 (see in FIG. 4 right side), which indication is also the operational instruction OI or a part of it. According to FIG. 4, the operational instruction OI, which indicates here exemplarily an absolute or relative change of the PCF of the product P1, P2, P3, may also indicate the corresponding PFC value x. Optionally, the operational instruction OI may also indicate by one or more graphical elements, as exemplarily illustrated in FIG. 4 by a respective arrow, whether the PFC of the product P1, P2, P3 changes in a positive direction or negative direction.

FIG. 5 illustrates in a flow chart a computer-implemented method for controlling production of products using a production process according to the present disclosure. The method may be carried out by the system 1 as described above and/or may be applied to the exemplary production processes shown in FIG. 3 or FIG. 4, in order to determine the at least one operational instruction OI configured to monitor and/or control the production process PP and/or the allocation of greenhouse emission among at least two different products to be produced with the production process PP.

In a step S100A, there is received production process data PPD comprising information about at least one process step PS, or the whole production process PP, producing at least two output materials, e.g. an intermediate I or the above products P1, P2, P3, within the production process PP. For example, the production process data PPD may be received by the processor 20 via the input interface 10, as described above.

In a step S100B, there is received at least information about a first allocation rule AL1, AL2, AL3 and a second allocation rule AL1, AL2, AL3 different to the first allocation rule AL1, AL2, AL3. In other words, there may be at least one allocation rule AL defining the distribution of greenhouse emission among the different products P1, P2, P3. For example, the allocation rule AL1, AL2, AL3 or information about that may be received by the processor 20 via the input interface 10, as described above. It is noted that the first allocation rule can also simply mean “one” allocation rule, and the second allocation rule may simply mean “another” allocation rule, which differs in at least one rule parameter from the “one” allocation rule.

It should be noted that the method steps S100A and S100B can be performed simultaneously or in any order.

In a step S200, there are determined, based on the production process data PPD, at least two products P1, P2, P3 affected by the first allocation rule AL1, AL2, AL3. Referring to FIG. 2, the products affected may be products P1 and P2, because the allocation Rule AL affects both products P1, P2, as it can be seen from the diagram. When referring to FIG. 3, the products affected may be the intermediates I1 and/or I2, or may be the products P1 and P2, because the allocation Rule AL affects both the intermediates I1, I2 and the products P1, P2, as it can be seen from the diagram. When referring to FIG. 3, the situation may be more complex, since multiple allocation rules AL1, AL2, AL3 may affect several of the intermediates I1-I5, several of the product steps PS1-PS6, and/or several of the products P1-P3.

Again referring to FIG. 5, in a step S300, there is determined, for the affected products P1, P2, P3, a first PCF PCF1 while applying the first allocation rule AL1, AL2, AL3 or the “one” allocation rule. This determination may be performed by the processor 20, as described above.

In a step S400, there is determined, for the affected products P1, P2, P3, a second PCF PCF2 while applying the second allocation rule AL1, AL2, AL3 or the “another” allocation rule. This determination may be performed by the processor 20, as described above.

In a step determining S500, there is determined, based on a comparison of the first PCF PCF1 and second PCF PCF2 with each other, an operational instruction OI. This determination may be performed by the processor 20, as described above.

In a step S600, there is outputted the determined operational instruction OI. This output may be performed by the output interface 30, as described above. The output may be used to monitor and/or control the production process, e.g. via a production process control, an ERP system or the like, as described above.

Further, the method may be modified in several ways as described above with regard to the system 1.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

1. A computer-implemented method for monitoring and/or controlling production of products using a production process in which an allocation rule is applied for allocating emissions contributing to the product carbon footprint, PCF, of the products among at least two different products, the method comprising:
receiving production process data comprising information about at least one process step producing at least two output materials within the production process;
receiving a first allocation rule and a second allocation rule different to the first allocation rule;
determining, based on the production process data, at least two products affected by the first allocation rule;
determining, for the affected products, a first PCF while applying the first allocation rule;
determining, for the affected products, a second PCF while applying the second allocation rule;
determining, based on a comparison of the first PCF and second PCF with each other, an operational instruction; and
outputting the determined operational instruction.

2. The method of claim 1, wherein the method is carried out prior to an actual change from the first to the second allocation rule within a production planning phase.

3. The method of claim 1, wherein different first and/or second allocation rules are received and/or applied for different process steps.

4. The method of claim 1, wherein a target PCF is specified for at least one of the at least two different products and the allocation rule suitable for achieving the target PCF is determined based on the determined operational instruction.

5. The method of claim 1, wherein the at least one process step is associated with multiple allocation rules, and the method further comprises:
comparing the second PCF to a PCF reference value associated with the product; and
determining a specific allocation rule among the multiple allocation rules that causes the PCF of the product to come closest to the PCF reference value.

6. The method of claim 1, wherein the at least one process step is associated with multiple allocation rules, and the method further comprises:
determining, based on the production process data, the products affected by the multiple allocation rules;
determining, for the affected products, a first PCF while applying an individual one of the multiple allocation rules;
determining, for the affected products, a second PCF while applying the individual one of the multiple allocation rules; and
determining, based on a comparison of the first PCF and second PCF with each other, for each determination applying the multiple allocation rules, the operational instruction.

7. The method of claim 1, further comprising:
applying an optimization algorithm utilizing a cost function to determine at least one allocation rule to be changed or replaced within the at least one process step and/or within the production process by another allocation rule that is determined by the optimization algorithm to decrease or minimize the PCF.

8. The method of claim 1, wherein the operational instruction is output via a user interface.

9. The method of claim 1, wherein information about the second allocation rule to be applied for determining the second PCF is received via a user interface configured to allow changing at least the allocation rule of the at least one process step.

10. The method of claim 1, further comprising:
replacing the first allocation rule by the second allocation rule if the determined operational instruction meets a replacement criterion.

11. The method of claim 1, wherein the first PCF and/or the second PCF is determined by:
receiving a carbon footprint of raw material used in the at least one process as input material;
receiving energy data comprising information about an energy consumption for the at least one process step; and
determining the first PCF and/or the second PCF of the product taking into account the production process data, the carbon footprint of raw material and/or the energy data.

12. A method for monitoring and/or controlling a production process comprising using an operational instruction determined by the method of claim 1.

13. A non-transitory computer readable data medium storing a computer program including instructions for executing steps of the method according to claim 1.

14. A system for monitoring and/or controlling production of products using a production process in which an allocation rule is applied for allocating emissions contributing to the product carbon footprint, PCF, of the products among at least two different products, the system comprising:
an input interface, configured to receive production process data comprising information about at least one process step producing at least two output materials within the production process, and a first allocation rule and a second allocation rule different to the first allocation rule;
a processor, configured to:
determine, based on the production process data, at least two products affected by the first allocation rule;
determine, for the affected products, a first PCF while applying the first allocation rule;
determine, for the affected products, a second PCF while applying the second allocation rule;
determine, based on a comparison of the first PCF and second PCF with each other, an operational instruction;
an output interface, configured to output the determined an operational instruction.

15. The system of claim 14, wherein the output interface comprises a user interface at least configured to display the operational instruction for further processing.
